(19) United States
(12) Patent Application Publication
(10) Pub. No.: US 2026/0234551 A1
(43) Pub. Date: Aug. 13, 2026

(54) HUMAN PLURIPOTENT STEM CELL LINE GENETICALLY ENGINEERED TO CO-EXPRESS TUBB3 GENE AND FLUORESCENT REPORTER GENE

(71) Applicants: CATHOLIC KWANDONG UNIVERSITY INDUSTRY FOUNDATION, Gangneung-si, Gangwon-do (KR); SEOUL NATIONAL UNIVERSITY R&DB FOUNDATION, Seoul (KR)

(72) Inventors: Ji Ho JANG, Seoul (KR); Young Min CHOI, Seoul (KR); Jong Kwan JUN, Seoul (KR)

(21) Appl. No.: 19/153,981

(22) PCT Filed: Feb. 7, 2024
(86) PCT No.: PCT/KR2024/001788

§ 371 Date: Aug. 6, 2025

(30) Foreign Application Priority Data
Feb. 10, 2023 (KR) 10-2023-0017856

(51) Int. Cl. C12N 5/0735; C12N 5/0793; C12N 9/22; C12N 15/113; G01N 21/64; G01N 33/50

(52) U.S. Cl. CPC C12N 5/0606; C12N 5/0619; C12N 9/226; C12N 15/113; G01N 21/6486; G01N 33/5005; C12N 2310/20; C12N 2506/02; C12N 2510/00

(57) ABSTRACT

Provided is a genetically engineered human pluripotent stem cell line in which an insertion sequence including a nucleotide sequence of a fluorescent reporter gene is inserted following a stop codon of the TUBB3 gene, whereby the TUBB3 gene and the fluorescent reporter gene are co-expressed. Genetic manipulation is made to express the fluorescent reporter gene in response to the expression of the TUBB3 gene, a neural cell marker, so that when the cell line is differentiated into neural cells, the fluorescent signal is observed, thereby allowing for monitoring the differentiation process.

### TECHNICAL FIELD

The present invention relates to a pluripotent cell line genetically engineered to insert a reporter gene to monitor the expression of the TUBB3 gene, which is a neuronal cell marker.

### BACKGROUND ART

Stem cells are cells with the special ability (multipotentiality) to produce many different types of cells, and can regenerate cells in damaged body parts. The term ‘stem cells’ was derived from the word ‘stem’, meaning to originate, and in Japan, they are also called stem cells, meaning they are primordial cells or stems that come from roots.

It is generally agreed that a stem cell's potential dwindles as it progresses from a fertilized egg to an embryo and eventually to an adult organism. According to these properties of stem cells, fertilized egg cells are called totipotent, while embryonic stem cells/induced pluripotent stem cells are called pluripotent, and adult stem cells are called multipotent. In particular, totipotent cells are those capable of developing into a complete organism. Totipotent cells include cells from the early embryonic stage along with fertilized egg cells. Typically, stem cells with totipotency include embryonic stem cells obtained from the internal cell mass of a divided blastocyst, and also induced pluripotent stem cells, which are somatic cells reprogrammed to have pluripotency.

Since embryonic stem cells and induced pluripotent stem cells can differentiate into all body cells, much research has been conducted on differentiating these pluripotent stem cells into desired cell types for use in the treatment of diseases.

Neurological diseases, such as stroke, Alzheimer's, Parkinson's, and spinal cord injury, are characterized by abnormal nerve function resulting from neuronal damage. Since damaged neurons have limited regenerative capacity, there are currently no treatments that fundamentally address the underlying causes of these conditions, with the exception of cell therapies designed to replace the compromised neural cells. Consequently, extensive research is underway to differentiate stem cells into neural cells for use in cellular therapies. In such differentiation studies, vigilant monitoring of proper stem cell differentiation into neural cells is crucial.

Accordingly, the inventors of the present invention created a cell line by inserting a fluorescent reporter gene into the TUBB3 gene, which is known to be involved in the elongation and maturation of exons in neural cells, in order to monitor differentiation into neural cells.

### DISCLOSURE OF INVENTION

### Technical Problem

An object to be solved in the present invention is to provide a human pluripotent cell line for monitoring differentiation into neural cells.

### Technical Solution

In order to achieve the technical problem, in one embodiment of the present invention, a genetically engineered human pluripotent cell line is provided in which an insertion sequence including a nucleotide sequence of a fluorescent reporter gene is inserted after a stop codon of a TUBB3 gene, thereby co-expressing the TUBB3 gene and the fluorescent reporter gene.

In one embodiment, the fluorescent reporter may be selected from the group consisting of Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, Phi YFP, Phi YFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, HcRed, KeimaRed, mRasberry, mPlum, PS-CFP, Dendra2, Kaede, EosFP, and KikumeGR.

In one embodiment, the insertion sequence may include a T2A cleavage sequence, a fluorescent reporter gene sequence, and a neomycin resistance gene sequence.

In one embodiment, the insert sequence may comprise SEQ ID NO: 1.

In one embodiment, the T2A cleavage sequence enables TUBB3 and the fluorescent reporter to be synthesized into their respective proteins during protein translation.

In one embodiment, the genetic manipulation may be genetically manipulated by the CRISPR/Cas9 system.

In one embodiment, the human pluripotent cell line may be prepared by genetically engineering SNUhES3, which is a human embryonic stem cell line.

In one embodiment, the genetically engineered human pluripotent cell line may express fluorescence while being differentiated into neural cells.

In addition, in an embodiment of the present invention, there is provided a method for monitoring differentiation into neural cells using the genetically engineered human pluripotent cell line.

### Advantageous Effects

A cell line prepared according to one embodiment of the present invention may provide a method for monitoring whether differentiation into neural cells is progressing properly by checking the fluorescent signal of a fluorescent reporter during the differentiation process of neural cells.

The effects of the present invention are not limited to the effects described above, and should be understood to include all effects that can be inferred from the constitution of the invention described in the Detailed Description or Claims of the present invention.

### BRIEF DESCRIPTION OF THE DRAWINGS

FIG. 1 shows a structure of the donor construct and the gene sequence targeted by the sgRNA of the CRISPR/Cas9 system for genetic manipulation of cell lines.

FIG. 2 shows the results of PCR analysis of genomic DNA (gDNA) of the SNUe003-A-4 cell line.

FIG. 3 shows the results of genetic sequence analysis of the SNUe003-A-4 cell line.

FIG. 4 shows the results of confirming the shape and AP activity of the SNUe003-A-4 cell line.

FIG. 5 shows the results of confirming the expression of pluripotency markers OCT4, SSEA-4, TRA-1-60, and TRA-1-81 in the SNUe003-A-4 cell line through fluorescent staining. This shows the results of a test to determine the presence/absence of mycoplasma infection.

FIG. 6 shows the results of RT-PCR for the expression of pluripotency markers OCT4, SOX2, and NANOG in the SNUe003-A-4 cell line.

FIG. 7 shows the results of karyotype analysis of the SNUe003-A-4 cell line.

FIG. 8 shows the results of testing for the presence/absence of mycoplasma infection in the SNUe003-A-4 cell line.

FIG. 9 shows the results of confirming the expression of germ layer-specific markers through fluorescent staining after inducing differentiation of SNUe003-A-4 cell line into the three germ layers.

FIG. 10 shows the results of confirming whether or not off-target genetic manipulation has occurred in the SNUe003-A-4 cell line.

FIG. 11 shows the results of confirming whether or not mCherry is expressed after differentiating the SNUe003-A-4 cell line into neural cells.

FIG. 12 shows the results of RT-PCR analysis to determine whether or not TUBB3 and MAP2 are expressed in cells expressing mCherry after differentiating the SNUe003-A-4 cell line into neural cells.

### BEST MODE FOR CARRYING OUT THE INVENTION

Hereinafter, the present invention will be described in detail.

According to one aspect of the present invention, a genetically engineered human pluripotent cell line is provided in which an insertion sequence including a nucleotide sequence of a fluorescent reporter gene is inserted after a stop codon of a TUBB3 gene, whereby the TUBB3 gene and the fluorescent reporter gene are co-expressed.

The above TUBB3 gene is a gene encoding the Tuj1 protein, and is a gene that begins to express in the early stage of differentiation of neural cells and plays an important role in the elongation and maturation of exons. Therefore, by confirming the expression of the TUBB3 gene, it is possible to monitor whether or not differentiation into neural cells is occurring properly.

The fluorescent reporter refers to a fluorescent substance used to report whether or not a specific gene is expressed. Therefore, general fluorescent materials may be used as reporters. For example, the reporter may be selected from the group consisting of Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, HcRed, KeimaRed, mRasberry, mPlum, PS-CFP, Dendra2, Kaede, EosFP, and KikumeGR, but is not limited thereto. Preferably, mCherry, a red fluorescent molecule, may be used.

The insertion sequence including the fluorescent reporter may be inserted after the stop codon of the TUBB3 gene in order to monitor the differentiation of neural cells. In such a case, the fluorescent reporter gene and the TUBB3 gene may be expressed as the same mRNA. In order to synthesize TUBB3 and the fluorescent reporter as their respective proteins, an IRES sequence and a 2A system sequence such as F2A, P2A, and T2A cleavage sequence may be positioned between the stop codon and the fluorescent reporter gene sequence, but is not limited thereto. Preferably, a T2A cleavage sequence may be used. 1. Through this, the TUBB3 gene and the fluorescent gene may be simultaneously expressed without affecting the TUBB3 function even after genetic manipulation.

In addition to the fluorescent reporter gene and the cleavage sequence, an antibiotic resistance gene for selecting a genetically engineered cell line may be inserted into the insertion sequence. For example, neomycin, puromycin, and hygromycin may be used, but are not limited thereto. Preferably, a neomycin resistance gene may be used.

For example, since cell line selection through the neomycin resistance gene is performed before the differentiation into neural cells, the neomycin resistance gene may use an independent promoter that may induce expression in pluripotent stem cells rather than being induced by the promoter of the TUBB3 gene. For example, CMV, U6, E2F, EF1-alpha, and PGK promoters may be selected, but are not limited thereto. Preferably, the PGK promoter may be used.

The insertion sequence may include SEQ ID NO: 1.

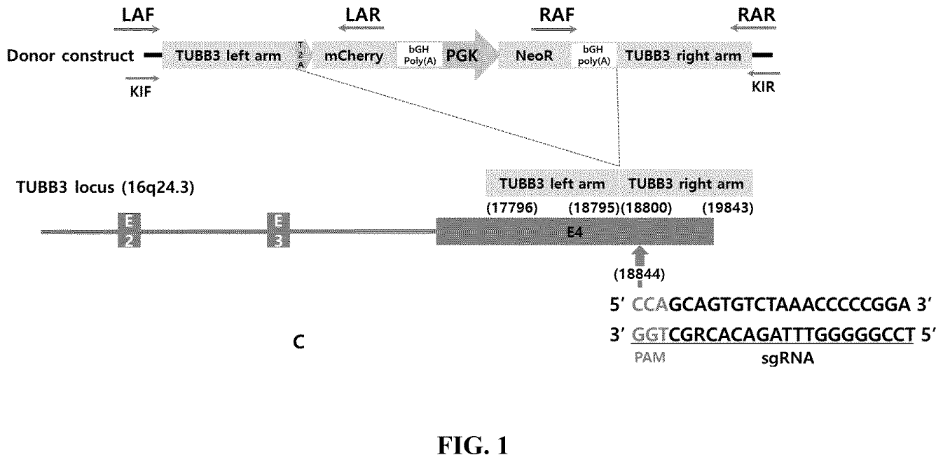

The genetic manipulation may be performed using the CRISPR/Cas9 system, which is gene-editing scissors. The CRISPR/Cas9 system may be composed of a Cas9 plasmid, sgRNA, and a donor construct. The sgRNA targets the 4th exon of the TUBB3 gene, and the donor construct may be composed of an insertion sequence including a fluorescent reporter gene within the homologous arm sequences on both sides of the TUBB3 gene. This is diagrammed in FIG. 1.

The Cas9 plasmid, sgRNA, and donor construct may be delivered into human pluripotent cells. The intracellular delivery method may include, but is not limited to, electroporation, lipofection, viral vectors, nanoparticles, and protein translocation domain (PTD) fusion protein methods. Preferably, electroporation may be used.

The genetically engineered human pluripotent cell line may be prepared by genetically engineering a human embryonic stem cell line or human induced pluripotent stem cell line. Preferably, the human embryonic stem cell line SNUhES3 may be used.

In addition, according to another aspect of the present invention, a method for monitoring differentiation into neural cells using the genetically engineered human pluripotent cell line may be provided.

For example, when the genetically engineered human pluripotent cell line is differentiated into neural cells, TUBB3 is expressed in the early stage of neural cell differentiation, and a fluorescent reporter may be expressed accordingly. This makes it possible to check in real time through a fluorescence microscope whether or not the differentiation is proceeding well, and to select only cells that are proceeding well using a flow cytometer.

Hereinafter, the present invention will be described in more detail through examples and test examples. However, the following examples and test examples are intended to illustrate the present invention, and the scope of the present invention is not limited thereto.

### Modes for Carrying Out the Invention

### EXAMPLES

### 1. Cell Culture

Human embryonic stem cell line, SNUhES3, was cultured in Essential 8 (E8, Gibco) medium under feeder-free conditions on vitronectin-coated plates. Subculture was performed at a 1:20 ratio after cell detachment using accutase.

### 2. Preparation of TUBB3-mCherry Reporter Cell Line

SNUhES3 cells (1×10⁵) were electroporated with the donor construct, Cas9 (Streptococcus pyogenes Cas9, ToolGen) plasmid, and sgRNA (ToolGen) plasmid using a Neon electroporator (Thermo fisher scientific). The gRNA targets the 4th exon of the TUBB3 gene, and the donor construct includes a T2A cleavage sequence, an mCherry gene sequence, and a neomycin resistance gene sequence between the two homologous arm sequences. This is shown in FIG. 1.

After electroporation, cells were plated on vitronectin-coated plates using E8 medium supplemented with Y-27632. After culturing for about 2 days, cells were treated with neomycin and selected for 3 weeks. The neomycin concentration was initially started at 50 μg/mL and increased to 150 μg/mL. After selection, 40 colonies resistant to neomycin were isolated, individually cultured, cryopreserved, and some were used for PCR analysis. For PCR analysis, genomic DNA (gDNA) was extracted from each colony and was subjected to PCR. gDNA was extracted using the DNeasy blood & Tissue kit (Qiagen), and PCR was performed using TaKaRa LA TaqR DNA polymerase (TAKARA Bio Inc.). Primer positions are shown in FIG. 1 and the sequences are shown in Table 1.

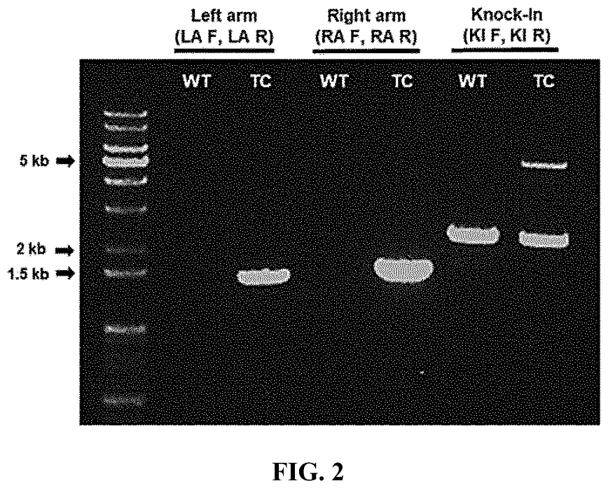

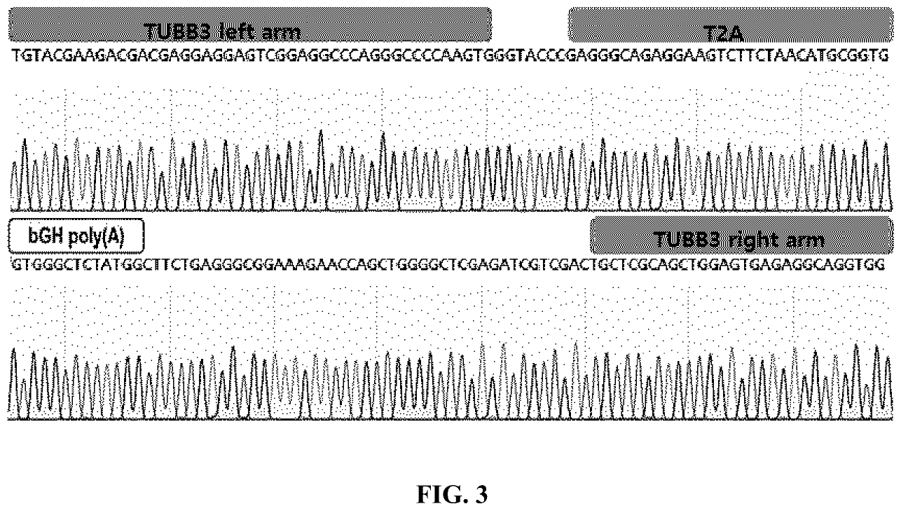

Six clones, which were genetically modified on only one chromosome, were selected through PCR. Then, one clone with an exact matching sequence was selected through DNA sequence analysis of the six clones. The PCR results are shown in FIG. 2, and the DNA sequence analysis results are shown in FIG. 3. Through this, the SNUe003-A-4 cell line was prepared in which the mCherry gene was inserted into the TUBB3 gene.

### 3. SNUe003-A-4 Cell Line Validation

It was confirmed whether or not the SNUe003-A-4 cell line maintained its pluripotency and was not damaged during the genetic manipulation process, and that mCherry was expressed when TUBB3 was expressed during the process of its differentiation into neural cells.

### 3-1) Confirmation of Maintenance of Pluripotency

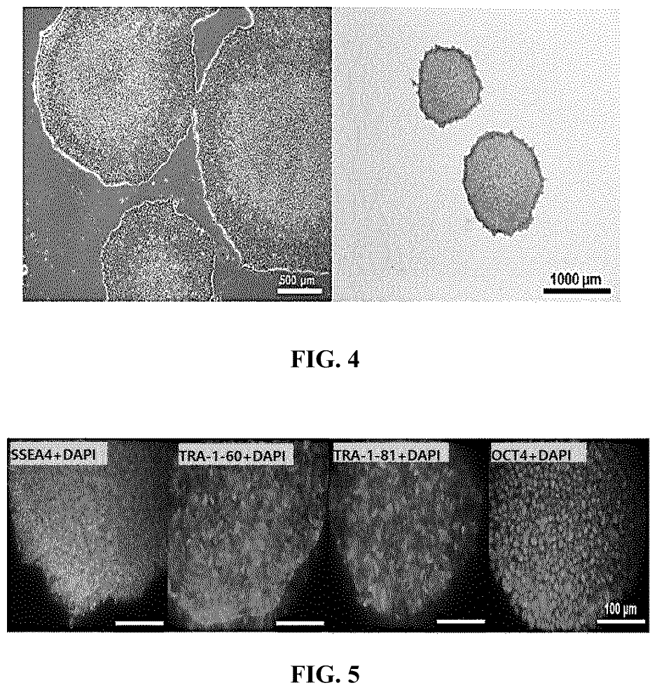

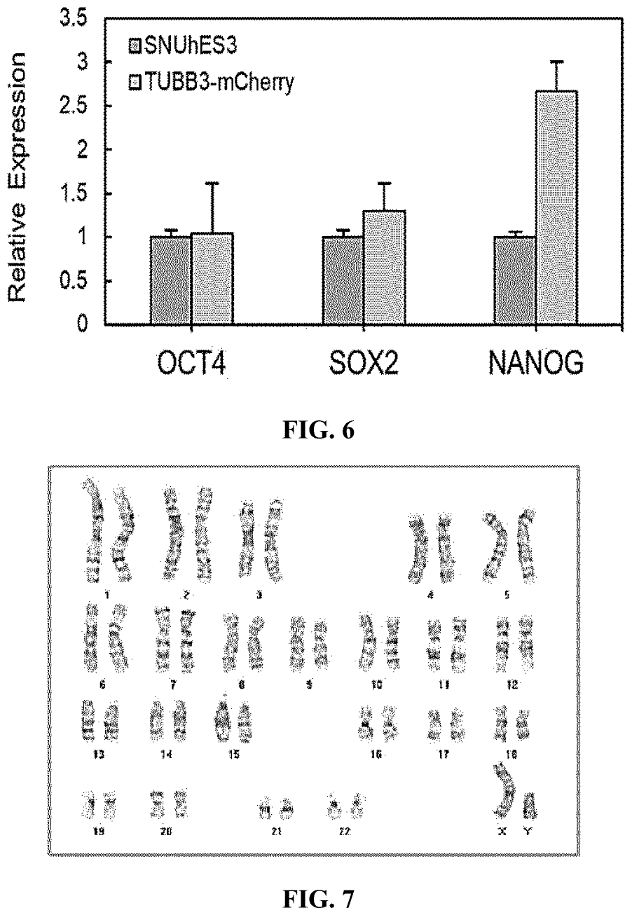

The SNUe003-A-4 cell line showed a typical human embryonic stem cell morphology, and AP activity was also confirmed. The results are shown in FIG. 4. In addition, the expression of pluripotency markers OCT4, SSEA-4, TRA-1-60, and TRA-1-81 was confirmed through fluorescent staining, and the results are shown in FIG. 5. The expression of pluripotency markers OCT4, SOX2, and NANOG was confirmed through RT-PCR, and the results are shown in FIG. 6. These results confirmed that the pluripotency of the SNUe003-A-4 cell line was not damaged during the genetic manipulation process.

### 3-2) Karyotype Analysis and STR Analysis

Karyotype analysis was performed using a standard protocol for GTG banding, and STR analysis was performed through Dowgene Co., Ltd. The results are shown in FIG. 7, and it was confirmed that the karyotype was intact.

### 3-3) Mycoplasma Contamination Analysis

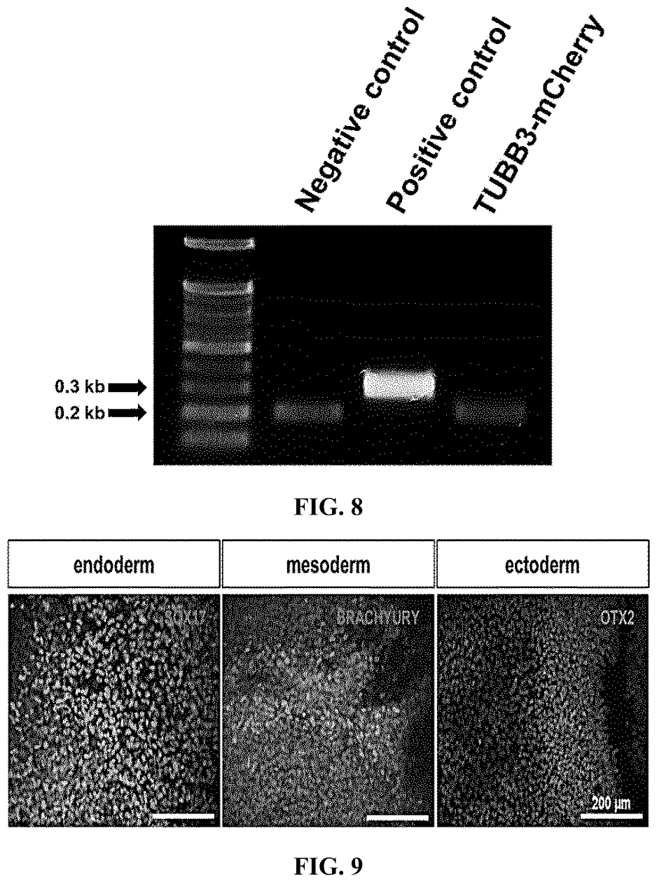

The presence/absence of mycoplasma contamination was confirmed using the e-Myco™ Mycoplasma PCR analysis kit from Intron Biotechnology, and the results are shown in FIG. 8. It was confirmed that there was no mycoplasma contamination in the SNUe003-A-4 cell line.

3-4) Experiment of Differentiation into Three Germ Layers

In order to confirm whether or not the SNUe003-A-4 cell line may differentiate into each of the three germ layers, the cell line was differentiated into the three germ layers using the Human Pluripotent Stem Cell Functional Identification kit (R&D system), and the expression of markers of each germ layer was confirmed through immunofluorescence staining. It was confirmed that the endoderm marker SOX17, the mesoderm marker BRACHYURY, and the ectoderm marker OTX2 were expressed. The results are shown in FIG. 9.

### Off-Target Analysis

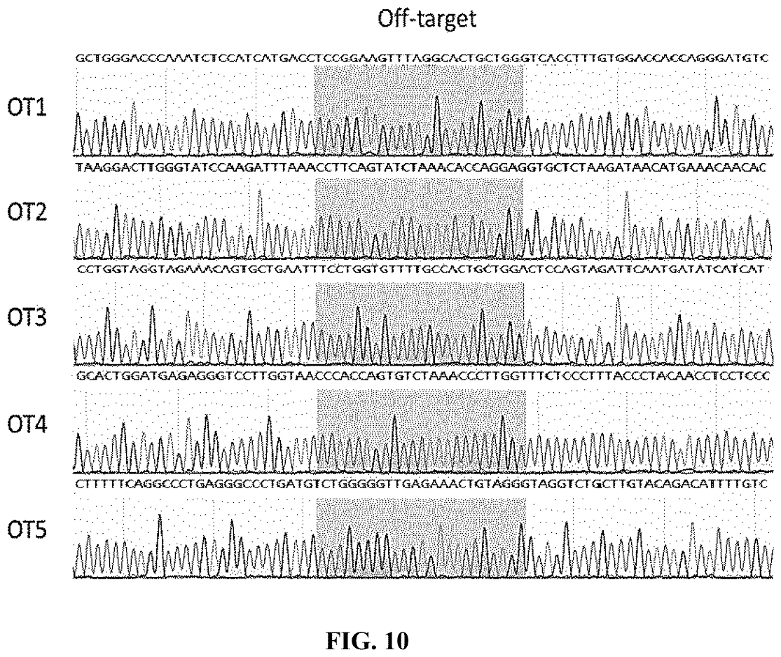

An experiment was performed to determine whether or not genetic modification occurred in off-targets other than TUBB3. First, five off-targets (OT1, OT2, OT3, OT4, and OT5) with a high possibility of genetic modification were derived using the CRISPR design tool (http:/CRISPR.mit.edu/). Then, sequence analysis was performed using Cosmogenetech Inc. after PCR of the corresponding parts in gDNA. The results are shown in FIG. 10. The sequence analysis results showed that no genetic modification occurred in the off-targets.

3-6) Differentiation into Neural Cells

It was confirmed whether or not TUBB3 was expressed simultaneously with mCherry when the SNUe003-A-4 cell line was differentiated into neural cells.

In order to differentiate into neural cells, the cell line was first made into embryoid bodies. The formed embryoid bodies were then attached to a culture dish and cultured for 5 days in DMEM/F12 (Gibco) medium containing N-2 supplement (Gibco), 20 μg/mL of insulin (Gibco), and 20 ng/ml of human bFGF (PEPROTECH) to induce differentiation into rosettes. Then, only the rosettes were transferred to culture dishes coated with Matrigel (Sigma) and cultured in DMEM/F12 (Gibco) medium containing 20 ng/ml of bFGF and 10 ng/ml of retinoic acid to induce the differentiation into neural progenitor cells (NPCs).

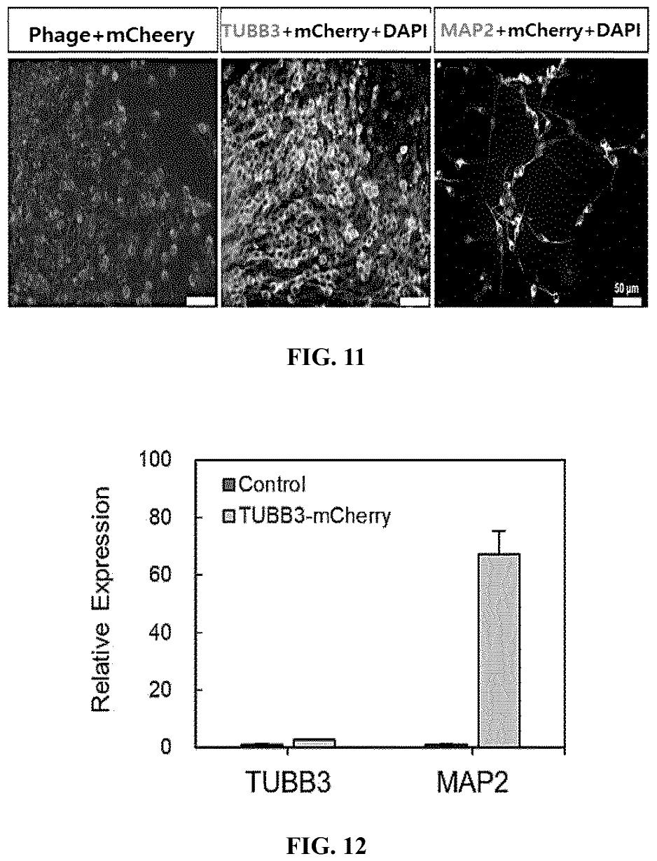

In order to confirm whether or not TUBB3 and MAP2, other neuronal markers, were expressed together with mCherry after inducing the differentiation into neural cells, fluorescent immunohistochemistry was performed, and the results are shown in FIG. 11. It was confirmed that mCherry was co-localized with TUBB3 and MAP2.

In addition, after the differentiation into neurons, it was confirmed through RT-PCR that mCherry-expressing neural cells had higher expression levels of TUBB3 and MAP2 compared to the control group. The results are shown in FIG. 12.

Through this, it was confirmed that it is possible to monitor the differentiation of neural cells because TUBB3 is expressed while the SNUe003-A-4 cell line is differentiated into neural cells, and thereby the mCherry reporter is expressed.

The antibodies used for fluorescent staining and the kit used for AP staining are provided in Table 1.

TABLE 1

| | Dilution | |
|---|---|---|
| Antibody/Kit | Ratio | Purchasing Company |
| Rabbit anti-OCT3/4 | 1:100 | Santa Cruz, Cat#sc-9081 |
| Mouse anti-SSEA4 | 1:100 | Millipore, MAB4304 |
| Mouse anti-TRA-1-60 | 1:100 | Millipore, MAB4360 |
| Mouse anti-TRA-1-81 | 1:100 | Millipore, MAB4381 |
| Alkaline phosphatase | | Sigma-Aldrich, Cat#86R- |
| | | 1KT |
| TUBB3 | 1:200 | Biolegend, CAT#801202 |
| MAP2 | 1:200 | Santa Cruz, CAT#sc- |
| | | 74421 |
| Goat anti-human OTX2 | 1:200 | R&D Systems, |
| | | Cat#AF1979 |
| Goat anti-human BRACHYURY | 1:200 | R&D Systems, |
| | | Cat#AF2085 |
| Goat anti-human SOX17 | 1:200 | R&D Systems, |
| | | Cat#AF1924 |
| Goat anti-human SOX17 | 1:200 | Thermo Fisher Scientific, |
| | | Cat#A11058 |
| Alexa Fluor 488 donkey anti- | 1:200 | Thermo Fisher Scientific, |
| goat IgG | | Cat#A11055 |
| Alexa Fluor 594 donkey anti- | 1:200 | Thermo Fisher Scientific, |
| rabbit IgG | | Cat#A21207 |
| Alexa Fluor 488 donkey anti- | 1:200 | Thermo Fisher Scientific, |
| mouse IgG | | Cat#A21202 |
| Alexa Fluor 488 donkey anti- | 1:200 | Thermo Fisher Scientific, |
| mouse IgG | | Cat#A21203 |
| DAPI | | Thermo Fisher Scientific, |
| | | Cat#D1306 |

The sequence information used in the experiment is provided in Table 2.

TABLE 2

| | Target Gene | Forward/Reverse Primer |
|---|---|---|
| Pluripotency | Nanog | TTTGTGGGCCTGAAGAAAACT/ |
| Markers | (116 bp) | AGGGCTGTCCTGAATAAGCAG |
| (qPCR) | Sox2 | AACCCCAAGATGCACAACTC/ |
| | (152 bp) | CGGGGCCGGTATTTATAATC |
| | OCT | CTACAACGCCTA |
| | (219 bp) | CGAGTCCTACA/ |
| | | TTCTGGCGCCGGTTACAGAAC |
| | | CA |
| Neuron | TUBB3 | TCAGCGTCTACTACAACGAGG |
| Markers | (120 bp) | C/ |
| (qPCR) | | GCCTGAAGAGATGTCCAAAGG |
| | | C |
| | MAP2 | AGGCTGTAGCAGTCCTGAAAG |
| | (153 bp) | G/ |
| | | CTTCCTCCACTGTGACAGTCTG |
| House- | GAPDH | GGAGCGAGATCCCTCCAAAAT/ |
| keeping | (197 bp) | GGCTGTTGTCATACTTCTCATG |
| Gene | | G |
| (qPCR) | | |
| Genotyping | KI (4.8 kb), | CCAAGGGTCACTACACGGAG/ |
| (desired | WT (2.1 kb) | CAGGTTGAAGGGTGATTC |
| allele/transgene | LA | CCAAGGGTCACTACACGGAG/ |
| presence | (1.4 kb) | GAGGAGTCCTGGGTCACGGTC |
| detection) | RA (1.5 kb) | CTGGATTCATCGACTGTG/ |
| | | CAGGTTGAAGGGTGATTC |
| Targeted | Junction | CAGCAGATGTTCGATGCCAAG/ |
| mutation | | GAGGAGTCCTGGGTCACGGTC |
| Analysis/ | | |
| sequencing | | |
| Potential | Plasmid backbond | GGTCCTGCAACTT |
| Random | | TATCCGCCTCCA/ |
| integration- | | AACCATGAGTGA |
| detecting PCR | | TAACACTGCGGCC |
| sgRNA | sgRNA target | TCCGGGGGTTTAGACACTGCT |
| | sequence | GG |
| Genomic | TUBB3 | NCBI reference sequence |
| target | | (NG_027810.1) Exon4 |
| sequence | | |
| Top off target | OT1 | TCGTCGGCAGCGTCGGG |
| Mutagenesis | (WSCD2\|CMKLR1) | CTTGACTGTGCTCATCT/ |
| predicted site | (399 bp) | GTCTCGTGGGCTCGGAGC |
| sequencing | | AACAAGGGCATCCTACC |
| (for | OT2 | TCGTCGGCAGCGTCATG |
| CRISPR/Cas9 | (AC104000.1\| | TAGGGTTAGGGGTGGCT/ |
| Primers) | RNA5SP261) | GTCTCTGGGGCTCGGGGGCC |
| | (422 bp) | TCTCAGAGCAAACAGT |
| | OT3 (RP11- | TCGTCGGCAGCGTCGGC |
| | 328K2.1\|TOX/RP11- | CTGGGCTTCTTCTGATT/ |
| | 25K19.1) | GTCTCGTGGGCTCGGTC |
| | (449 bp) | AGGCAGTTTCCTCAGCTC |
| | OT4 (IL23R) | TCGTCGGCAGCGTCAAG |
| | (491 bp) | TTTCCCCATCACCACCC/ |
| | | GTCTCGTGGGCTCGGAA |
| | | ACCAGGTGGCTGCTTAGG |
| | OT5 (MTX1/RP11- | TCGTCGGCAGCGTCACT |
| | 263K19.6\|GBA) | AGCCCAACCAGTGCATC/ |
| | (477 bp) | GTCTCGTGGGCTCGGCGG |
| | | ACTCAGGCTCAGAAAGG |

The description of the present invention is for illustrative purposes, and those skilled in the art to which the present invention pertains will understand that the present invention may be easily modified into other specific forms without changing the technical idea or essential characteristics of the present invention. Therefore, it should be understood that the embodiments described above are exemplary in all respects and not restrictive. For example, each component described as a single component may be implemented in a distributed manner, and likewise, components described as distributed may be implemented in a combined manner.

The scope of the present invention is indicated by the claims set forth below, and all changes or modifications derived from the meaning and scope of the claims and the equivalent concepts thereof should be interpreted as being included in the scope of the present invention.

### INDUSTRIAL APPLICABILITY

The present invention relates to a genetically engineered human pluripotent cell line in which an insertion sequence including a fluorescent reporter gene sequence is inserted after a stop codon of the TUBB3 gene, so that the TUBB3 gene and the fluorescent reporter gene are co-expressed. The cell line is prepared by inserting a fluorescent reporter gene into the TUBB3 gene, which is known to be involved in the elongation and maturation of exons in neural cells, in order to monitor differentiation into neural cells. The present invention provides a method for monitoring whether or not differentiation into neural cells is progressing properly by confirming the fluorescent signal of the fluorescent reporter during the differentiation process of neural cells, and thus may be utilized in various ways, such as in the treatment of neurological diseases, such as stroke, Alzheimer's disease, Parkinson's disease, and spinal cord injury.

Accordingly, it may be seen that a human pluripotent cell line genetically engineered to co-express the TUBB3 gene and the fluorescent reporter gene according to one embodiment of the present invention has industrial applicability.

1. A genetically engineered human pluripotent cell line in which an insertion sequence comprising a nucleotide sequence of a fluorescent reporter gene is inserted following a stop codon of the TUBB3 gene, whereby the TUBB3 gene and the fluorescent reporter gene are co-expressed.

2. The genetically engineered human pluripotent cell line according to claim 1, wherein the fluorescent reporter is selected from the group consisting of Sirius, EBFP, ECFP, m Turquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, Turbo YFP, Zs Yellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, HcRed, KeimaRed, mRasberry, mPlum, PS-CFP, Dendra2, Kaede, EosFP, and KikumeGR.

3. The genetically engineered human pluripotent cell line according to claim 1, wherein the insertion sequence comprises a T2A cleavage sequence, a fluorescent reporter gene sequence, and a neomycin resistance gene sequence.

4. The genetically engineered human pluripotent cell line according to claim 3, wherein the insertion sequence comprises SEQ ID NO: 1.

5. The genetically engineered human pluripotent cell line according to claim 3, wherein TUBB3 and the fluorescent reporter are synthesized into respective proteins during protein translation by the T2A cleavage sequence.

6. The genetically engineered human pluripotent cell line according to claim 1, wherein the genetic manipulation is genetically engineered by the CRISPR/Cas9 system.

7. The genetically engineered human pluripotent cell line according to claim 1, wherein the human pluripotent cell line is prepared by genetically engineering SNUhES3 that is a human embryonic stem cell line.

8. A genetically engineered human pluripotent cell line according to claim 1, wherein the human pluripotent cell line expresses fluorescence while being differentiated into neural cells.

9. A method for monitoring the differentiation of the genetically engineered human pluripotent cell line according to claim 1 into neural cells.


SEQUENCE LISTING (1 sequence)

SEQ ID NO: 1 - DNA, 2715 bp, synthetic construct
  source 1..2715: mol_type = other DNA
ggtacccgagggcagaggaagtcttctaacatgcggtgacgtggaggagaatcccggccctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggccgcgccggctctagatcgcgaacgcgtgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctcaatatgattgaacaagatggattgcacgcaggttctccggccgcttgggtggagaggctattcggctatgactgggcacaacagacaatcggctgctctgatggcgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtaactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctccccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatcgtcgac